(19) United States
(12) Patent Application Publication
(10) Pub. No.: US 2008/0069899 A1
(43) Pub. Date: Mar. 20, 2008

(54) Pharmaceutical Compositions Comprising Beta-Carboline Derivatives and Use Thereof for the Treatment of Cancer

(76) Inventors: Akino Jossang born Yanagida, Paris (FR); Georges Uzan, Vitry Sur Seine (FR); Jean Kenji Jossang, Paris (FR)

Correspondence Address: YOUNG & THOMPSON, 745 SOUTH 23RD STREET, 2ND FLOOR, ARLINGTON, VA, 22202, US

(21) Appl. No.: 11/587,852

(22) PCT Filed: Apr. 29, 2005
(86) PCT No.: PCT/FR05/01082

§ 371 Date: Oct. 15, 2007

(30) Foreign Application Priority Data
Apr. 30, 2004 (FR) 04/04646

(51) Int. Cl. A61K 31/437; A61K 31/5025; A61K 31/513; A61K 31/661; A61K 33/24; A61P 35/00

(52) U.S. Cl. 424/649; 514/110; 514/249; 514/274; 514/292; 514/83

(57) ABSTRACT

The invention relates to the use of at least one compound of general formula (1) for the production of a medicament for the treatment of cancer.

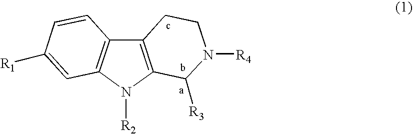

The present invention relates in particular to pharmaceutical compositions containing β-carboline derivatives, an alkaloid, corresponding in particular to those extracted from Peganum harmala, such as harmine, harman and harmalacidine, and their use within the framework of the treatment of cancer.

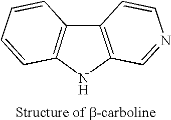

Peganum harmala, a North African or Asian herb, is used in traditional medicine for numerous disorders. Peganum alkaloids are known for their antibacterial, antifungal, antiviral, hypothermic properties and above all their hallucinogenic effects. (Boukef, “Les plantes dans la médecine traditionnelle tunisienne”, Agence de Coopération Culturelle et Technique, Paris 1986)

An earlier study has demonstrated an antineoplastic activity of extracts of P. harmala seeds on rat and mouse tumors. Thus, the administration of an alkaloid crude extract of P. harmala seeds, at a dose of 50 mg/kg/day, by oral route, made the (subcutaneous) grafted tumors in mice disappear in 80% of the mice treated (Lamchouri et al. (1999) Thérapie 54: 753-758).

Moreover, certain purified and isolated P. harmala alkaloids have shown a moderate cytotoxicity vis-à-vis murine tumorous cells, with a concentration inhibiting 50% of cell growth (IC₅₀) of 19.2 to 60 μg/ml for vasicinone, 2.4 to 18.4 μg/ml for harmine, inactive for peganine and 8.0 to 28.9 μg/ml for harmalacidine (Lamchouri, thesis “Propriétés cytotoxiques et antitumorales de Peganum harmala sur des modèles expérimentaux de cancer in vitro et in vivo” (2000) Faculté des Sciences Dhar Mahraz, Fès, Maroc).

Finally, another study has also demonstrated a moderate cytotoxic action of certain β-carboline derivatives, such as harmine (IC₅₀ of 1.6 to 18.5 μg/ml) and harman (IC₅₀ of 8 to 20 μg/ml) on certain human tumor cell lines (Ishida et al. (1999) Bioorg. Med. Chem. Lett. 9: 3319-3324).

In general it is considered for a given compound that an IC₅₀ of less than 0.01 μg/ml on human cells is necessary in order to be able to envisage moving to in vivo tests in mice.

Moreover, there is no mention in the prior art of any antineoplastic effect in vivo of β-carboline derivatives, corresponding in particular to those extracted from P. harmala.

Moreover, it should be noted that the demonstration of a cytotoxic activity of a given compound vis-à-vis tumor cell lines, even human cell lines, does not at all imply an antineoplastic effect in vivo. In fact, this compound can for example prove to be inactive in vivo or even have a cytotoxicity such that it is incompatible with its administration to a living organism.

Similarly, compounds which are active on murine tumors are not necessarily active on human cancer.

Therefore a subject of the present invention is to provide pharmaceutical compositions comprising compounds derived from β-carboline for the preparation of medicaments intended for the treatment of cancer.

A subject of the present invention is also to provide another compound capable of entering into synergy with the compounds derived from β-carboline for the preparation of medicaments intended for the treatment of cancer.

Thus, the present invention relates to the use of at least one compound of general formula (1) 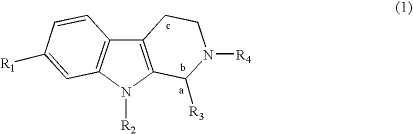
in which:
R₁ represents H, OH, or an alkoxyl group with 1 to 12 carbon atoms,
R₂ represents H, an alkoxycarbonyl group with 1 to 12 carbon atoms, in particular the tert-butoxycarbonyl group, or an alkyl group with 1 to 12 carbon atoms,
R₃ represents O or CH₃, providing that, when R₃ represents O, then a represents a double bond, b and c represent a single bond and R₄ represents H, and that when R₃ represents CH₃ then a represents a single bond, b and c represent a double bond and R₄ does not represent any group;
   or its pharmaceutically acceptable salts, for the preparation of a medicament intended for the treatment of cancer, such as colon cancer, leukemia, myelomas, breast cancer, neuroblastomas, hepatocarcinomas, lung cancer, prostate cancer, ovarian cancer, testicular cancer, gastric cancer, pancreatic cancer, or retinoblastomas.

According to a particular embodiment of the invention, at least one compound of general formula (1) is combined with at least one compound inhibiting the replication of DNA.

By “compound inhibiting DNA replication” is meant any compound capable of inhibiting a stage of DNA replication, whether by inhibiting the activity of the enzymes involved in the replication, such as DNA polymerases, topoisomerases, helicases, primases, ligases, or by binding or by modifying DNA, for example by binding to the two strands of DNA (such as alkylating agents), or by preventing synthesis of thymidine.

Advantageously, the combination of a compound of general formula (1) with a compound inhibiting DNA replication has synergistic effects in the treatment of tumors. Moreover, the compound of general formula (1) is advantageously used as an adjuvant intended to increase the effects of compounds inhibiting DNA replication within the framework of the preparation of anti-tumor medicaments.

As meant here, the term “combination” signifies that the compound of general formula (1) and the compound inhibiting replication of DNA are both present in a structurally independent manner in a medicament or a pharmaceutical composition according to the invention and that they are not bound to each other by strong chemical bonds of a covalent or coordination type.

Among the pharmaceutically acceptable salts of the compounds of general formula (1), the hydrochloride salts are particularly preferred.

According to a more particular embodiment of the invention, the compound of general formula (1) corresponds:
to the compounds of formula (2) below, 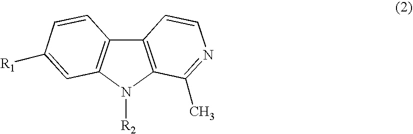
or to the compounds of formula (3) below, 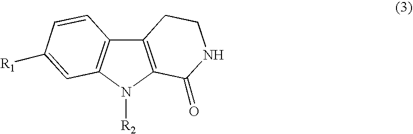
   in which R₁ and R₂ are as defined above.

According to an even more particular embodiment of the invention, the compound of general formula (1) corresponds to:

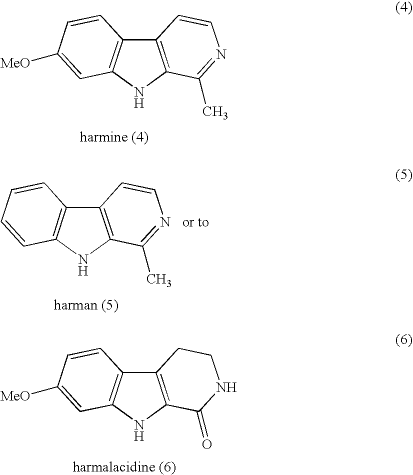

Advantageously, the Inventors have demonstrated that harmine as well as harmalacidine had an anti-tumor action in vivo, in particular on tumors in humans. This action is reinforced when harmine and harmalacidine are administered in combination with a compound inhibiting DNA replication.

Advantageously, the pharmaceutically acceptable salts of the compounds of formulae (4), (5) and (6) above can also be used according to the invention, and in particular harmine hydrochloride, harman hydrochloride and harmalacidine hydrochloride.

Moreover, it has been shown that harmane, the chemical structure of which is close to that of harmine, possesses a common target cell with harmine (Sobhani et al. (2002) J. Pharm. Pharmaceut. Sci. 5: 19-23). It can therefore be used for the same purpose as harmine within the framework of the invention.

According to another particular embodiment of the invention, the compound inhibiting DNA replication is chosen from the group comprising:
an alkylating agent, such as cyclophosphamide, mitomycin C or thiotepa;
an antimetabolite, such as 5-fluorouracil, ara C or methotrexate;
a coordination complex of platinum such as carboplatin or cisplatin;
or an agent inhibiting topoisomerase II, such as doxorubicin, mitoxantrone or amsacrine.

These compounds are well known to a person skilled in the art.

In particular, an alkylating agent acts by preventing the separation of the two strands of DNA of a single fragment by producing a solid covalent bridging.

An antimetabolite prevents this, either by taking the place of the bases (5-fluorouracil or ara-C), or by inhibiting enzymatic synthesis of the thymidine (5-fluorouracil, methotrexate).

The coordination complexes of platinum in particular produce a solid bridging between the two strands of DNA in a single fragment.

Topoisomerase II has the activity of cleaving and rejoining the two strands of DNA of a single fragment within the framework of the relaxation of supercoiled DNA.

According to a preferred embodiment of the invention, the latter relates to the use as defined above, in which the molar quantity of the compound of general formula (1) is greater than the molar quantity of the compound inhibiting the replication of the DNA with which it is combined. In particular, the molar quantity of the compound of general formula (1) is at least 20% greater than the molar quantity of the compound inhibiting the replication of the DNA with which it is combined.

According to a preferred embodiment of the invention, the latter relates to the use as defined above of harmine or harmalacidine, and cyclophosphamide.

According to another preferred embodiment of the invention, the latter relates to the use as defined above of harmine or harmalacidine, and 5-fluorouracil.

The present invention also relates to a pharmaceutical composition comprising as active ingredient at least one compound of general formula (1), or one of its pharmaceutically acceptable salts, in combination with at least one compound inhibiting the replication of DNA and a pharmaceutically acceptable vehicle.

According to a preferred embodiment of the invention, the pharmaceutical composition is such that the compound of general formula (1) corresponds to:

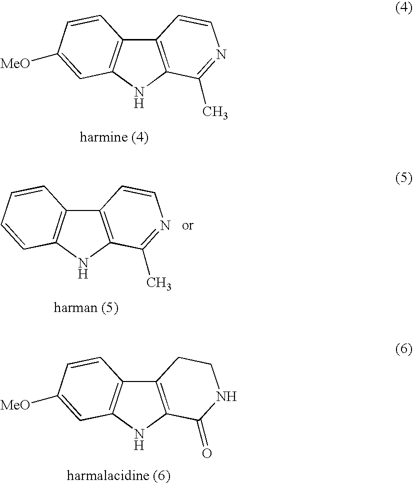

According to another preferred embodiment of the invention, the pharmaceutical composition is such that the compound inhibiting the DNA replication is chosen from the group comprising:
an alkylating agent, such as cyclophosphamide, mitomycin C or thiotepa;
an antimetabolite, such as 5-fluorouracil, ara C or methotrexate;
a coordination complex of platinum, such as carboplatin or cisplatin;
or an agent inhibiting topoisomerase II, such as doxorubicin, mitoxantrone or amsacrine.

According to another preferred embodiment, the pharmaceutical composition according to the invention is suitable for administration by oral or intravenous route.

According to a particularly preferred embodiment, the pharmaceutical composition according to the invention comprises as active ingredient harmine or harmalacidine, in combination with cyclophosphamide and a pharmaceutically acceptable vehicle.

According to a more particularly preferred embodiment, the abovementioned pharmaceutical composition is suitable for administration by oral route:
of approximately 1 to approximately 10 mg/kg/day of harmine, or approximately 1 to approximately 5 mg/kg/day of harmalacidine, and approximately 1 to approximately 5 mg/kg/day of cyclophosphamide.

According to another more particularly preferred embodiment, the abovementioned pharmaceutical composition is suitable for administration by intravenous route:
approximately 1 to approximately 3 mg/kg/day of harmine, or approximately 1 to approximately 3 mg/kg/day of harmalacidine, and approximately 1 to approximately 5 mg/kg/day of cyclophosphamide.

According to another particularly preferred embodiment, the pharmaceutical composition according to the invention comprises as active ingredient harmine or harmalacidine, in combination with 5-fluorouracil and a pharmaceutically acceptable vehicle.

According to a more particularly preferred embodiment, the abovementioned pharmaceutical composition is suitable for administration by oral route:
approximately 1 to approximately 10 mg/kg/day of harmine, or approximately 1 to approximately 5 mg/kg/day of harmalacidine, and approximately 1 to approximately 10 mg/kg/day of 5-fluorouracil.

According to another more particularly preferred embodiment, the abovementioned pharmaceutical composition is suitable for administration by intravenous route:
of approximately 1 to approximately 3 mg/kg/day of harmine, or approximately 1 to approximately 3 mg/kg/day of harmalacidine, and approximately 1 to approximately 5 mg/kg/day of 5-fluorouracil.

According to a preferred embodiment, the pharmaceutical composition as defined above is suitable for the administration of a molar quantity of the compound of general formula (1) greater than the molar quantity of the compound inhibiting the replication of the DNA with which it is combined. In particular, the pharmaceutical composition is suitable for the administration of a molar quantity of the compound of general formula (1) at least 20% greater than the molar quantity of the compound inhibiting the replication of the DNA with which it is combined.

The present invention also relates to products containing
at least one compound of general formula (1), or one of its pharmaceutically acceptable salts, and
at least one compound inhibiting DNA replication,
   as combination products for simultaneous or separate use, or spread over time within the framework of the treatment of cancer, such as colon cancer, leukemia, myelomas, breast cancer, neuroblastomas, hepatocarcinomas, lung cancer, prostate cancer, ovarian cancer, testicular cancer, gastric cancer, pancreatic cancer, or retinoblastomas.

As meant here, the term “combination” signifies that the compound of general formula (1) and the compound inhibiting the DNA replication are both present in a structurally independent manner in the products according to the invention and that they are bound to each other by strong chemical bonds of the covalent or coordination type.

According to a particular embodiment of the invention, the combination products are such that the compound of general formula (1) corresponds to:

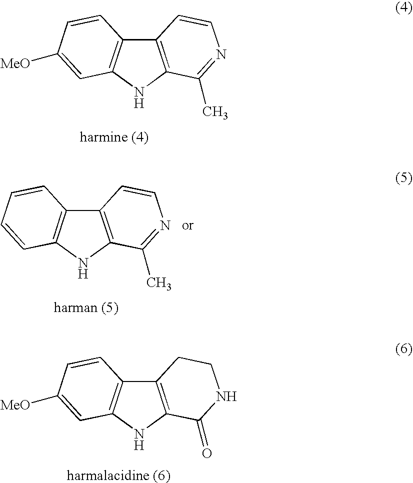

According to another particular embodiment of the invention, the combination products are such that the compound inhibiting the replication of DNA is chosen from the group comprising:
an alkylating agent, such as cyclophosphamide, mitomycin C or thiotepa;
an antimetabolite, such as 5-fluorouracil, ara C or methotrexate;
a coordination complex of platinum, such as carboplatin or cisplatin;
or an agent inhibiting topoisomerase II, such as doxorubicin, mitoxantrone or amsacrine.

According to a preferred embodiment, the invention relates to products as defined above, containing
harmine or harmalacidine, and
5-fluorouracil,
   as combination products for a simultaneous or separate use, or spread over time within the framework of the treatment of cancer, such as colon cancer, breast cancer, hepatocarcinomas, lung cancer, prostate cancer, ovarian cancer, gastric cancer, or pancreatic cancer.

According to another preferred embodiment, the invention relates to products as defined above, containing:
harmine or harmalacidine, and
cyclophosphamide,

as combination products for simultaneous or separate use, or spread over time within the framework of the treatment of cancer, such as colon cancer, leukemia, myelomas, breast cancer, neuroblastomas, hepatocarcinomas, lung cancer, ovarian cancer, testicular cancer, or retinoblastomas.

According to a preferred embodiment, the products as defined above include a molar quantity of the compound of general formula (1) greater than the molar quantity of the compound inhibiting the replication of the DNA with which it is in combination. In particular, the products include a molar quantity of the compound of general formula (1) at least 20% greater than the molar quantity of the compound inhibiting the replication of the DNA with which it is in combination.

### DESCRIPTION OF THE FIGURES

FIG. 1

FIG. 1 represents the size development of HT29 tumors (in cm³, y axis) grafted onto NOD-SCID mice, untreated (diamonds), or treated with 100 mg/kg/day (crosses), 125 mg/kg/day (squares), 150 mg/kg/day (triangles) or 175 mg/kg/day (circles) of harmine as a function of time (in days, x axis).

FIG. 2A and FIG. 2B

FIG. 2A represents the size development of HT29 tumors (in cm³, y axis) grafted onto NOD-SCID mice, untreated (crosses), or treated with 50 mg/kg/day (triangles), 100 mg/kg/day (squares) of cyclophosphamide, or with a mixture of 100 mg/kg/day of harmine+100 mg/kg/day of cyclophosphamide (circles) or 150 mg/kg/day of harmine+50 mg/kg/day of cyclophosphamide (diamonds) as a function of time (in days, x-axis).

FIG. 2B represents the size development of HT29 tumors (in cm³, y axis) grafted onto NOD-SCID mice, untreated (diamonds), or treated with 150 mg/kg/day (triangles) of harmine, 50 mg/kg/day (crosses) of cyclophosphamide, or with a mixture of 150 mg/kg/day of harmine+50 mg/kg/day of cyclophosphamide (circles) as a function of time (in days, x axis).

FIG. 3A and FIG. 3B

FIG. 3A represents the size development of HT29 tumors (in cm³, y axis) grafted onto NOD-SCID mice, untreated (circles), or treated with 3 mg/kg/day (triangles), 6 mg/kg/day (squares), 9 mg/kg/day (crosses), 12 mg/kg/day (diamonds) or 24 mg/kg/day (dashes)) of 5-fluorouracil as a function of time (in days, x axis).

FIG. 3B represents the growth of HT29 tumors (in cm³, y axis) grafted onto NOD-SCID mice, untreated (triangles), or treated with 150 mg/kg/day of harmine (crosses), 12 mg/kg/day of 5-fluorouracil (squares), or with a mixture of 150 mg/kg/day of harmine+12 mg/kg/day of 5-fluorouracil (circles) as a function of time (in days, x axis).

FIG. 4

FIG. 4 represents the growth of HT29 tumors (in cm³, y axis) grafted onto NOD-SCID mice, untreated (squares), or treated with 25 mg/kg/day (triangles) or 50 mg/kg/day (crosses) of harmalacidine, with 50 mg/kg/day of cyclophosphamide (diamonds), or with a mixture of 25 mg/kg/day of harmalacidine+50 mg/kg/day of cyclophosphamide (dashes) or 50 mg/kg/day of harmalacidine+50 mg/kg/day of cyclophosphamide (circles), as a function of time (in days, x axis).

### EXAMPLES

### Example 1

Extractions of Alkaloids of Peganum harmala

Powder from ground P. harmala seeds (Zygophyllaceae) (1 kg) was extracted with methanol. After evaporation of the solvent, the residue was solubilized in 2% hydrochloric acid. The acidic aqueous solution was then washed with dichloromethane, then alkalized with sodium bicarbonate and extracted with dichloromethane. The organic phase then leaves a crude extract of a mixture of the alkaloids (24 g) by evaporation of the solvent. The extract is subjected to chromatography on a silica column eluting with a dichloromethane/methanol (9/1) mixture and separated into 23 fractions.

The crystallization of fractions 3 to 5 in a dichloromethane/methanol mixture provides pure harmine (A) (3 g) (M: 212.3; MP.261° C. (261° C.; Goebel, F. Justus Liebigs Ann. Chem. 1841, 38, 363 and Hochstein, A. J. Amer. Chem. Soc., 1957, 49, 5735)). Vasicine (B) is extracted from fractions 10 to 12, harmalacidine (C) (12.6 g, M:216, MP. 197° C. (197-198° C.; Hashimoto, Y., Kawanishi, K. Phytochemistry 1976, 15, 1559-1560; Siddiqui, S. Heterocycles 1988, 27, 1401)) from fractions 17 to 21 and demethyl harmalacidine (D) from fractions 22 to 23.

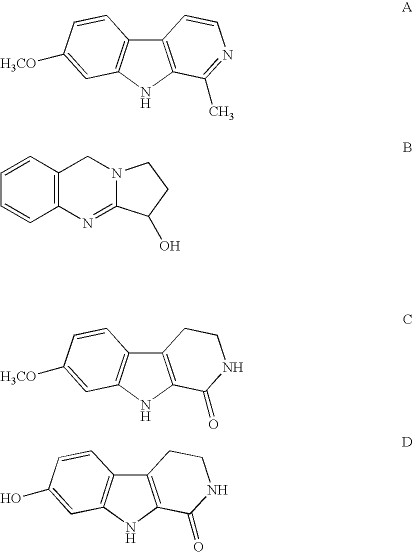

The harmine was precisely characterized:
the mass spectrum of harmine shows the molecular ion [M]^{.+} with m/z 212 Dalton, corresponding to the molecular formula C₁₃H₁₂N₂O (M: 212.3).
analysis of the NMR ¹H and ¹³C spectra and of the 2-dimensional COSY, HSQC and HMBC spectra confirms the structure of harmine given below. 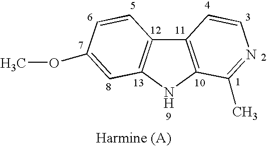
dihydrated harmine hydrochloride was also analyzed; MP. 262° C. (268-270° C.; The Merck Index, Xth, M. Windholz, ed., Merck 1 co., Inc., Rahway, N.J. USA, 1983, p. 666), M. 284.8;
the toxicity was determined in BALB/c mice; lethal dose 50 (LD₅₀) 300 mg/kg per os. (243 mg/kg, sc, mice, 38 mg/kg, iv, The Merck Index, Xth, M. Windholz, ed., Merck 1 co., Inc., Rahway, N.J. USA, 1983, p. 666).

The harmalacidine was also characterized:
the mass spectrum of harmalacidine shows the molecular ion [M]^{.+} with m/z 216 Dalton, corresponding to the molecular formula C₁₂H₁₂N₂O₂ (M: 216.2).
analysis of the NMR ¹H and ¹³C spectra and of the 2-dimensional COSY, HSQC and HMBC spectra confirms the structure of harmalacidine given below (Hashimoto, Y., Kawanishi, K. Phytochemistry 1976, 15, 1559-1560; Siddiqui, S. Heterocycles 1988, 27, 1401). 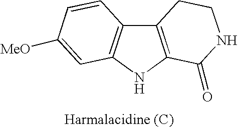
the molecular weight of dihydrated harmalacidine hydrochloride: M 288.

### Example 2

Cytotoxic Effects of the Alkaloids of P. harmala In Vitro

The cytotoxic effects of the different alkaloid extracts of P. harmala were studied on several cell lines:
human leukemia cell lines: K562 and Jurkat (leukemia), U937 (myeloma),
human solid tumor cell lines: KB (epithelioma of the nasopharynx) and HT29 (colon),
immortalized human bone marrow endothelial cell lines: HBMEC.

The cells are cultured in an oven under a 5% CO₂ atmosphere and at 37° C., in RPMI 1640 medium for K562, Jurkat, U937 and HT29 and DMEM for KB, supplemented with 10% foetal calf serum, 0.01% penicillin-streptomycin and L-glutamine 2 mM and in EGM2 medium for the HBMEC cells.

The cytotoxicity test is carried out in a 96-well microplate in the presence of the extract to be tested at varying concentrations of 40, 20, 10, 5, 1, 0.5 μg/ml and in the absence of product, after incubation for 4 days at 37° C. On the 3^{rd} day, a solution of neutral red is added which is absorbed by living cells. The optical density (OD) of dye released by lyzed cells is measured at 540 nm by an Elisa plate reader. The toxicity (% inhibition of growth) is inversely proportional to the optical density.

The percentage of inhibition is defined as being the difference between the OD without product and the OD in the presence of product compared to the OD without product.

The concentration inhibiting 50% of cell growth is obtained from the curve representing the percentage of inhibition as a function of the logarithm of the concentration.

All of the results obtained are shown in Table 1 below.

TABLE 1
in vitro cytotoxicity of the alkaloids of Peganum harmala

| | IC₅₀in μg/ml:μM | | | | | |
|---|---|---|---|---|---|---|
| Cell line | KB | K562 | Jurkat | U937 | HT29 | HBMEC |
| Crude extract | 5.3:— | 11.8:— | 6.3:— | 10.2:— | 2.7:— | 8.0:— |
| Harmine (A) | 4.6:21.7 | 3.5:16.5 | 3.2:15.1 | 3.5:16.5 | 2.9:13.7 | 3.5:16.5 |
| Harmine•HCl•2H₂O | 8.5:29.8 | 9.7:34.1 | 5.0:17.6 | 7.2:25.3 | 3.1:10.9 | 3.2:11.2 |
| Harmalacidine (C) | 12:55.5 | 12.5:57.8 | 15.5:71.7 | 29:134 | 10.5:34.4 | 28:130 |
| Demethyl- | 10:49.5 | 20:99 | 8.5:42 | N.D. | N.D. | 17:84.1 |
| harmalacidine (D) | | | | | | |
| Peganine (B) | >50:266 | 35:186 | 75:399 | N.D. | N.D. | N.D. |
| Camptothecin | 0.035:0.1 | 0.014:0.04 | 0.003:0.09 | 0.023:0.07 | N.D. | 0.025:0.07 |

To summarize, the IC₅₀ vis-à-vis malignant and endothelial cells tested is the following:

5 to 12 μg/ml for the crude extract of alkaloids,

2.9 to 4.6 μg/ml (14-22 μM) for harmine,

3 to 10 μg/ml (10.5-35 μM) for harmine hydrochloride, and

10.5 to 29 μg/ml (34.4-134 μM) for harmalacidine hydrochloride.

### Example 3

In Vivo Antineoplastic Activity of Harmine

Materials and Methods

The animal model chosen is that of severe combined immunodeficient (NOD-SCID) mice having received a xenograft of human tumor cells.

Male and female NOD-SCID mice, more than 3 months old, were raised in an environment of strict sterility, in an insulator ventilated with filtered air and sterilized, at 22° C. and 40% humidity, with a day-12 h/night-12 h cycle. The cages, feeding bottles and the water were sterilized in an autoclave at 120° C. for 30 minutes and the food as well as the bedding were treated with γ-irradiation. All handling took place in aseptic conditions under a laminar flow hood.

The mice were subjected to general anaesthesia by i.p. injection of 0.3 to 0.4 ml of hypnomidate at 2 mg/ml. 1×10⁷ HT29 human colon tumor cells in suspension in 200 μl of PBS were then injected sub-cutaneously, into the backs of the mice. On the 10th day of graft the length of the tumor reaches approximately 1 cm.

201 mg of harmine hydrochloride were dissolved in 8 ml of water and the solution obtained was sterilized by filtration on a membrane with 0.22 μm porosity.

On the tenth day following the graft, harmine was administered by oral route (per os) using a stomach tube at a rate of 0 (control), 100, 125, 150, 175 and 200 mg/kg/day for 60 days respectively to 6 groups of 5 mice.

The approximate volume of the tumor was regularly calibrated and calculated according to the formula:
Volume(cm³)=length(cm)×width(cm)×height(cm)×0.5
Results

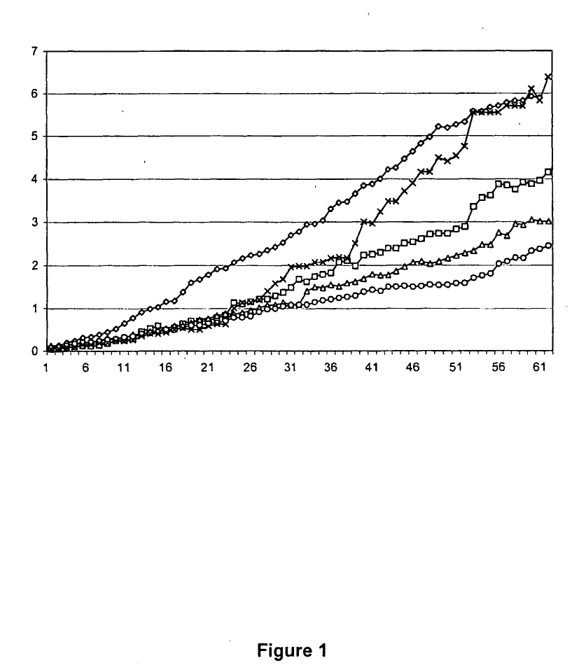

The results obtained are shown in Table 2 and in FIG. 1.

TABLE 2
treatment with harmine of mice xenografted with HT29 human tumour cells

| | | Average volume of the tumours of the treated mice | | | |
|---|---|---|---|---|---|
| | | compared to the average volume of the tumours of | | | |
| | Dose of harmine | the non-treated mice (T/C) (%) | | | |
| Group | (mg/kg/day) | Day 30 | Day 40 | Day 50 | Day 60 |
| 1 | 0 | 100 | 100 | 100 | 100 |
| 2 | 100 | 74 | 77 | 84 | 105 |
| 3 | 125 | 55 | 58 | 54 | 67 |
| 4 | 150 | 41 | 46 | 42 | 51 |
| 5 | 175 | 40 | 10 | 30 | 40 |
| 6 | 200 | — | — | — | — |

The inhibitory action of harmine on the growth of the tumors manifests itself in a dose-dependent manner between 125 to 175 mg. The dose of 100 mg/kg/day has a very weak effect. The dose of 125 mg/kg/day begins to show a significant effect: the volume of the tumor reaches 55 to 67% of the volume of the tumour of the non-treated group. At the dose of 150 mg/kg/day, the volume of the tumor represents only 42% of that of the control group until day 50, but reaches 51% on the 60^{th} day. The dose of 175 mg/kg/day has proved to be effective with T/C=40%, for the entire duration of treatment. The dose of 200 mg/kg/day was not tolerated, the mice died after a few days of treatment.

### Example 4

In Vivo Antineoplastic Activity of Cyclophosphamide, Alone or in Combination With Harmine

The antineoplastic activity of cyclophosphamide (M=261), alone or in combination with harmine, was measured according to the methodology of Example 3.

150 mg of cyclophosphamide (Sigma) were dissolved in 8 ml of water and the solution obtained was sterilized by filtration on a membrane with 0.22 μm porosity. The mice received either 50, 100, or 150 mg/kg/day of cyclophosphamide alone, or a mixture of 150 mg/kg/day of harmine+50 mg/kg/day of cyclophosphamide or 100 mg/kg/day of harmine+100 mg/kg/day of cyclophosphamide.

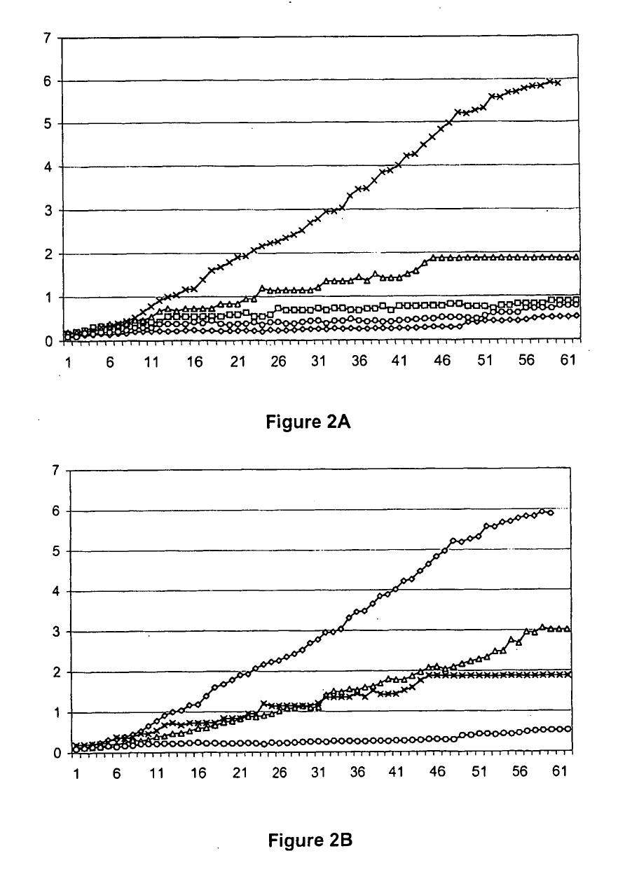

The results obtained are shown in Table 3 and in FIGS. 2A and 2B.

TABLE 3
Treatment with cyclophosphamide, alone or in combination with
harmine, of mice xenografted with HT29 human tumour cells

| | | | Average volume of the tumours of the treated mice | | | | |
|---|---|---|---|---|---|---|---|
| | Dose of | Dose of | compared to the average volume of the tumours of | | | | |
| | harmine | cyclophosphamide | the non-treated mice (T/C) (%) | | | | |
| Group | (mg/kg/day) | (mg/kg/day) | Day 15 | Day 30 | Day 40 | Day 50 | Day 60 |
| 1 | 0 | 0 | 100 | 100 | 100 | 100 | 100 |
| 2 | 0 | 50 | 63 | 42 | 37 | 36 | 32 |
| 3 | 0 | 100 | 47 | 25 | 18 | 14 | 15 |
| 4 | 0 | 150 | 4 | — | — | — | — |
| 5 | 150 | 50 | 20 | 10 | 7 | 8 | 9 |
| 6 | 100 | 100 | 36 | 16 | 11 | 10 | 9 |

When the cyclophosphamide is administered alone (Table 3, FIGS. 2A, 2B), the dose of 50 mg/kg/day leads to a T/C ratio of 40 to 30%. The dose of 100 mg/kg/day inhibited the tumor growth very strongly: T/C of 25 to 15%. At the dose of 150 mg/kg/day, the tumor volume has scarcely increased, but the mice did not tolerate this dose and all died around the 15^{th} day of treatment.

During the simultaneous administration of harmine at 150 mg with cyclophosphamide at 50 mg/kg/day (Table 3, FIG. 2B) (molar ratio harmine/cyclophosphamide 3.7/1), the tumor volume remained stationary with a T/C ratio less than 10% until the 60^{th} day of the treatment. The harmine-cyclophosphamide combination therefore has a synergistic effect on the inhibition of tumor growth, compared to the use of harmine alone and of cyclophosphamide alone at the concentrations used in the mixture. This makes it possible to envisage the use of this combination for the treatment of cancer.

However, it should be noted that the combination of 150 mg/kg/day of harmine with 50 mg/kg/day of cyclophosphamide was not well tolerated from the 30th day. The symptom of cumulative intoxication is manifested in the yellowing of the fur or the appearance of edema as a result of a hepatorenal disorder, which disappear on stopping the cyclophosphamide. It was therefore necessary to stop the daily administration of cyclophosphamide and to switch to a cycle of 7 days of rest-3 days of treatment. The harmine by contrast was given without interruption. The mice subjected to this combined and controlled treatment show no sign of intoxication and 75% of the mice thus treated survived more than 100 days with a tumour volume of less than 1 cm³.

The combination of 100 mg/kg/day of harmine+100 mg/kg/day of cyclophosphamide produces a similar inhibition of the tumor growth, with a T/C ratio close to 10% (Table 3, FIG. 2A).

### Example 5

In Vivo Antineoplastic Activity of 5-fluorouracil, Alone or in Combination With Harmine

The antineoplastic activity of 5-fluorouracil (M=130), alone or in combination with harmine, was measured according to the methodology of Example 3.

50 mg of 5-fluorouracil (Sigma) were dissolved in 8 ml of water and the solution obtained was sterilized by filtration on a membrane with 0.22 μm porosity. The mice received either 3, 6, 9, 12, or 24 mg/kg/day of 5-fluorouracil alone, or a mixture of 150 mg/kg/day of harmine+12 mg/kg/day of 5-fluorouracil.

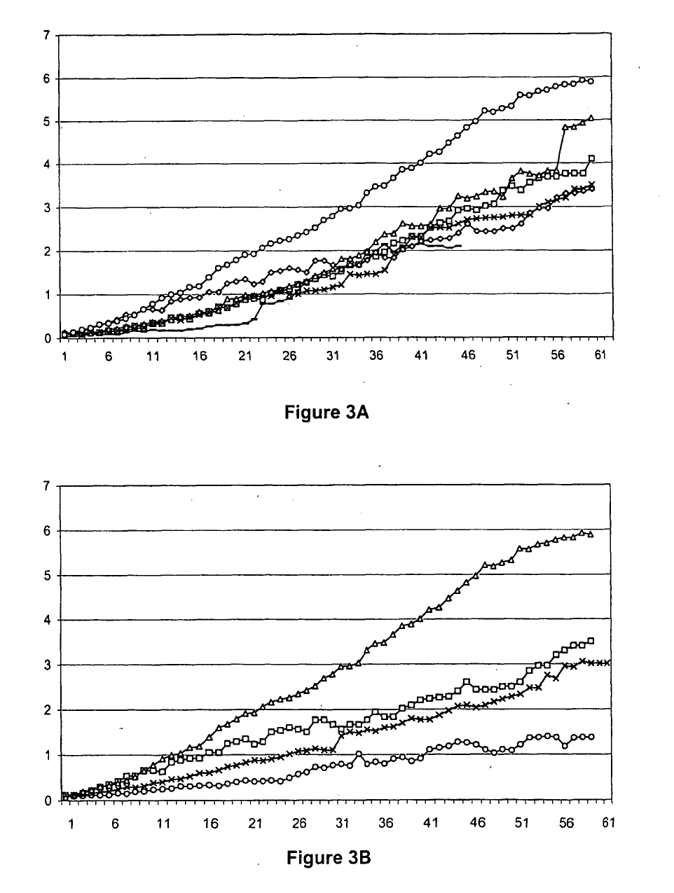

The results obtained are shown in Table 4 and in FIGS. 3A and 3B.

TABLE 4
Treatment with 5-fluorouracil, alone or in combination with
harmine, of mice xenografted with HT29 human tumour cells

| | | | Average volume of the tumours of the treated mice | | | | |
|---|---|---|---|---|---|---|---|
| | Dose of | Dose of | compared to the average volume of the tumours of | | | | |
| | harmine | 5-fluorouracil | the non-treated mice (T/C) (%) | | | | |
| Group | (mg/kg/day) | (mg/kg/day) | Day 15 | Day 30 | Day 40 | Day 50 | Day 60 |
| 1 | 0 | 0 | 100 | 100 | 100 | 100 | 100 |
| 2 | 0 | 3 | 43 | 56 | 66 | 61 | 86 |
| 3 | 0 | 6 | 37 | 53 | 60 | 64 | 70 |
| 4 | 0 | 9 | 41 | 41 | 59 | 52 | 59 |
| 5 | 0 | 12 | 73 | 66 | 54 | 47 | 59 |
| 6 | 0 | 24 | 17 | 54 | 54 | — | — |
| 7 | 150 | 12 | 25 | 26 | 22 | 21 | 23 |

When 5-fluorouracil is administered alone per os, at doses comprised between 3 and 12 mg/kg/day, for 60 days (Table 4 and FIG. 3A), the tumors (HT29) continue to grow. With the dose of 12 mg/kg/day, the size of the tumors increases slowly until Day 50 (T/C=52%), then the tumours begin to grow more rapidly, to reach at Day 60 a size similar to that observed with the dose of 9 mg/kg/d. The dose of 24 mg/kg/d, administered by alternating 6 days of treatment and 6 days of rest, is effective until the 20th day of the treatment (T/C=17%) but, thereafter, the effectiveness decreases: at the 30^{th} day (T/C=54%). This dose becomes lethal between the 35 and the 45^{th} day of administration.

Harmine at 150 mg/kg/day was administered simultaneously in combination with increasing doses of 5-fluorouracil: 3, 6, 9 and 12 mg/kg/day respectively.

Only the combination of 150 mg/kg/day of harmine+12 mg/kg/day of 5-fluorouracil is shown in Table 4 (harmine/5-fluorouracil molar ratio of 7.7/1). This combination, administered in a cycle of 6 days of treatment and 6 days of rest, has synergistic effects on the inhibition of the tumor growth (Table 4, FIG. 3B) and was able to keep the tumor volume below 1.3 cm even at the 60^{th} day of treatment, with 23% T/C.

### Example 6

In Vivo Antineoplastic Activity of Vinblastine, Alone or in Combination With Harmine

The antineoplastic activity of vinblastine is dose-dependent between 0.125 and 0.5 mg/kg/day until the 40th day, by sub-cutaneous administration. A dose greater than 1 mg/kg/day was not tolerated by the NOD-SCID mice beyond the 6^{th} day.

No synergistic effect was observed for the combinations of at 150 mg/kg/day of harmine and 0.125, 0.25 and 0.5 mg /kg/day of vinblastine respectively.

### Example 7

In Vivo Antineoplastic Activity of Harmalacidine, Alone or in Combination With Cyclophosphamide

The antineoplastic activity of harmalacidine, alone or in combination with cyclophosphamide, was measured according to the methodology of Example 3.

100 mg of dihydrated harmalacidine hydrochloride were dissolved in 8 ml of water and the solution obtained was sterilized by filtration on a membrane with 0.22 μm porosity. The mice received either 25, 50, or 100 mg/kg/day of harmalacidine alone, or a mixture of 25 or 50 mg/kg/day of harmalacidine+50 mg/kg/day of cyclophosphamide.

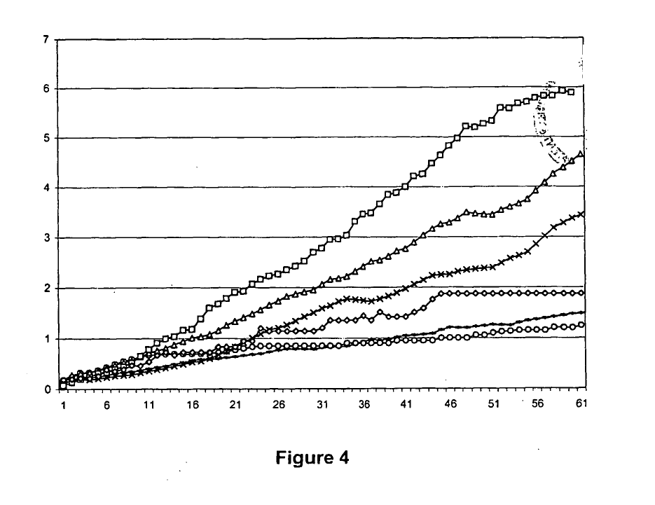

The results obtained are shown in Table 5 and in FIG. 4.

TABLE 5
Treatment with harmalacidine, alone or in combination with cyclophosphamide,
of mice xenografted with HT29 human tumour cells

| | | | Average volume of the tumours of the treated mice | | | | |
|---|---|---|---|---|---|---|---|
| | Dose of | Dose of | compared to the average volume of the tumours of | | | | |
| | harmalacidine | cyclophosphamide | the non-treated mice (T/C) (%) | | | | |
| Group | (mg/kg/day) | (mg/kg/day) | Day 15 | Day 30 | Day 40 | Day 50 | Day 60 |
| 1 | 0 | 0 | 100 | 100 | 100 | 100 | 100 |
| 2 | 25 | 0 | 81 | 72 | 70 | 65 | 77 |
| 3 | 50 | 0 | 42 | 56 | 48 | 45 | 57 |
| 4 | 100 | 0 | dead | — | — | — | — |
| 5 | 25 | 50 | 45 | 29 | 26 | 23 | 25 |
| 6 | 50 | 50 | 60 | 32 | 24 | 20 | 20 |

When harmalacidine is administered alone per os, at doses of 25 and 50 mg/kg/day, for 60 days (Table 5 and FIG. 4), the tumors (HT29) grow slowly up to Day 50 (T/C=65 and 45%), then begin to grow more rapidly, to reach T/C=77 and 57% at Day 60. The dose of 100 mg/kg/day was not tolerated for more than 10 days.

25 or 50 mg/kg/day of harmalacidine was administered simultaneously in combination with 50 mg/kg/day of cyclophosphamide.

This combination, administered in a cycle of 5 days of treatment and 2 days of rest, demonstrates synergistic effects on the inhibition of the tumor growth (Table 5, FIG. 4) and was able to keep the tumor volume below 1.5 and 1.2 cm³ even at the 60^{th} day of treatment, with respectively 25 and 20% T/C.

1-22. (canceled)

23. Pharmaceutical composition comprising as active ingredient at least one compound of general formula (1): 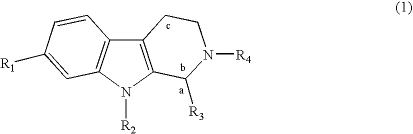
in which:
R₁ represents H, OH, or an alkoxyl group with 1 to 12 carbon atoms,
R₂ represents H, a alkoxycarbonyl group with 1 to 12 carbon atoms, in particular the tert-butoxycarbonyl group, or an alkyl group with 1 to 12 carbon atoms,
R₃ represents O or CH₃, providing that, when R₃ represents O, then a represents a double bond, b and c represent a single bond and R₄ represents H, and that when R₃ represents CH₃ then a represents a single bond, b and c represent a double bond and R₄ does not represent any group;
or one of its pharmaceutically acceptable salts, in combination with at least one compound inhibiting DNA replication and a pharmaceutically acceptable vehicle.

24. The pharmaceutical composition according to claim 23, in which the compound of general formula (1) corresponds to:

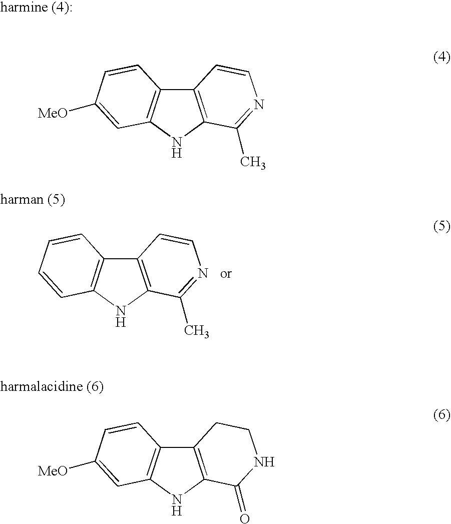

25. The pharmaceutical composition according to claim 23, in which the compound inhibiting DNA replication is chosen from the group comprising:
an alkylating agent, such as cyclophosphamide, mitomycin C or thiotepa;
an antimetabolite, such as 5-fluorouracil, ara C or methotrexate;
a coordination complex of platinum, such as carboplatin or cisplatin;
or an agent inhibiting topoisomerase II, such as doxorubicin, mitoxantrone or amsacrine.

26. The pharmaceutical composition according to claim 23, suitable for administration by oral or intravenous route.

27. The pharmaceutical composition according to claim 23, comprising harmine or harmalacidine as active ingredient, in combination with cyclophosphamide and a pharmaceutically acceptable vehicle.

28. The pharmaceutical composition according to claim 27, suitable for administration by oral route: of (approximately) 1 to (approximately) 10 mg/kg/day of harmine, or (approximately) 1 to (approximately) 5 mg/kg/day of harmalacidine, and (approximately) 1 to (approximately) 5 mg/kg/day of cyclophosphamide.

29. The pharmaceutical composition according to claim 27, suitable for administration by intravenous route: of (approximately) 1 to (approximately) 3 mg/kg/day of harmine, or (approximately) 1 to (approximately) 3 mg/kg/day of harmalacidine, and (approximately) 1 to (approximately) 5 mg/kg/day of cyclophosphamide.

30. The pharmaceutical composition according to claim 23, comprising harmine or harmalacidine as active ingredient, in combination with 5-fluorouracil and a pharmaceutically acceptable vehicle.

31. The pharmaceutical composition according to claim 30, suitable for administration by oral route: of (approximately) 1 to (approximately) 10 mg/kg/day of harmine, or (approximately) 1 to (approximately) 5 mg/kg/day of harmalacidine, and (approximately) 1 to (approximately) 10 mg/kg/day of 5-fluorouracil.

32. The pharmaceutical composition according to claim 30, suitable for administration by intravenous route: of (approximately) 1 to (approximately) 3 mg/kg/day of harmine, or (approximately) 1 to (approximately) 3 mg/kg/day of harmalacidine, and (approximately) 1 to (approximately) 5 mg/kg/day of 5-fluorouracil.

33. Products containing
at least one compound of general formula (1): 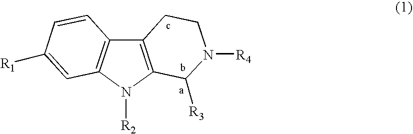
in which:
R₁ represents H, OH, or an alkoxyl group with 1 to 12 carbon atoms,
R₂ represents H, a alkoxycarbonyl group with 1 to 12 carbon atoms, in particular the tert-butoxycarbonyl group, or an alkyl group with 1 to 12 carbon atoms,
R₃ represents O or CH₃, providing that, when R₃ represents O, then a represents a double bond, b and c represent a single bond and R₄ represents H, and that when R₃ represents CH₃ then a represents a single bond, b and c represent a double bond and R₄ does not represent any group;
or its pharmaceutically acceptable salts, and
at least one compound inhibiting DNA replication, as combination products for simultaneous or separate use or spread over time within the framework of the treatment of cancer selected from the group consisting of: colon cancer, leukemia, myelomas, breast cancer, neuroblastomas, hepatocarcinomas, lung cancer, prostate cancer, ovarian cancer, testicular cancer, gastric cancer, pancreatic cancer and (or) retinoblastomas.

34. Products according to claim 33, in which the compound of general formula (1) corresponds to:

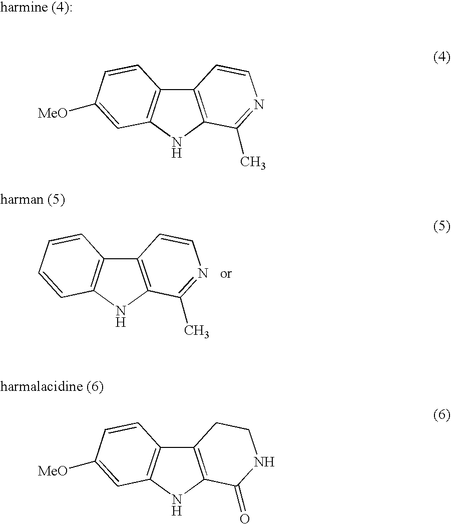

35. Products according to claim 33, in which the compound inhibiting DNA replication is chosen from the group comprising:
an alkylating agent, such as cyclophosphamide, mitomycin C or thiotepa;
an antimetabolite, such as 5-fluorouracil, ara C or methotrexate;
a coordination complex of platinum, such as carboplatin or cisplatin;
or an agent inhibiting topoisomerase II, such as doxorubicin, mitoxantrone or amsacrine.

36. Products according to claim 33, containing
harmine or harmalacidine, and
5-fluorouracil,
such as combination products for simultaneous or separate use or spread over time within the framework of the treatment of cancer selected from the group consisting of: colon cancer, breast cancer, hepatocarcinomas, lung cancer, prostate cancer, ovarian cancer, gastric cancer and (or) pancreatic cancer.

37. Products according to one of claims 33, containing:
harmine or harmalacidine, and
cyclophosphamide,
such as combination products for simultaneous or separate use or spread over time within the framework of the treatment of cancer selected from the group consisting of: colon cancer, leukemia, myelomas, breast cancer, neuroblastomas, hepatocarcinomas, lung cancer, ovarian cancer, testicular cancer, and (or) retinoblastomas.

38. A method for the treatment of cancer selected from the group consisting of: colon cancer, leukemia, myelomas, breast cancer, neuroblastomas, hepatocarcinomas, lung cancer, prostate cancer, ovarian cancer, testicular cancer, gastric cancer, pancreatic cancer, and (or) retinoblastomas, said method comprising the administration to a patient in need thereof, of a pharmaceutically acceptable amount of a compound of general formula (1): 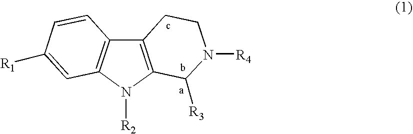
in which:
R₁ represents H, OH, or an alkoxyl group with 1 to 12 carbon atoms,
R₂ represents H, a alkoxycarbonyl group with 1 to 12 carbon atoms, in particular the tert-butoxycarbonyl group, or an alkyl group with 1 to 12 carbon atoms,
R₃ represents O or CH₃, providing that, when R₃ represents O, then a represents a double bond, b and c represent a single bond and R₄ represents H, and that when R₃ represents CH₃ then a represents a single bond, b and c represent a double bond and R₄ does not represent any group;
or its pharmaceutically acceptable salts.

39. The method according to claim 38, wherein said compound of general formula (1) is in combination with at least one compound inhibiting DNA replication.

40. The method according to claim 38, in which the compound of general formula (1) corresponds:
to the compounds of formula (2) below, 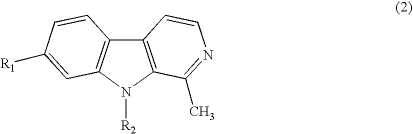
or to the compounds of formula (3) below, 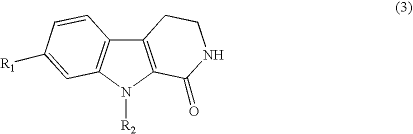
in which R₁ and R₂ are as previously defined.

41. The method according to claim 38, in which the compound of general formula (1) corresponds to:

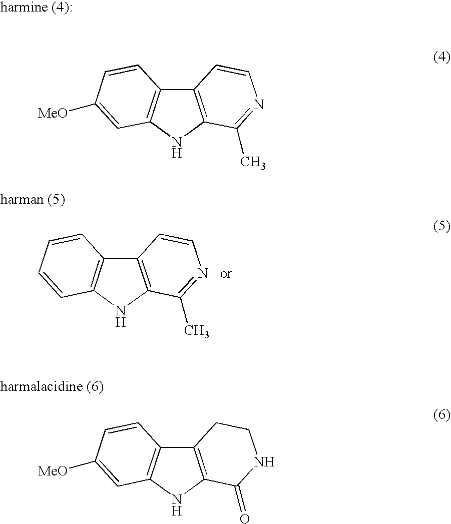

42. The method according to claim 39, in which the compound inhibiting DNA replication is chosen from the group comprising:
an alkylating agent, such as cyclophosphamide, mitomycin C or thiotepa;
an antimetabolite, such as 5-fluorouracil, ara C or methotrexate;
a coordination complex of platinum, such as carboplatin or cisplatin;
or an agent inhibiting topoisomerase II, such as doxorubicin, mitoxantrone or amsacrine.

43. The method according to claim 39, wherein harmine or harmalacidine is in combination with cyclophosphamide.

44. The method according to claim 39, wherein harmine or harmalacidine is in combination with 5-fluorouracil.
